(19) United States
(12) Patent Application Publication
(10) Pub. No.: US 2009/0068640 A1
(43) Pub. Date: Mar. 12, 2009

(54) METHODS FOR DETECTING AND INACTIVATING A PRION

(76) Inventors: Robert ROHWER, Ellicott City, MD (US); Luisa Gregori, Baltimore, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/269,479

(22) Filed: Nov. 12, 2008

(30) Foreign Application Priority Data
Jun. 27, 2003 (US) PCT/US2003/020065

(51) Int. Cl. C12Q 1/70; A61L 2/00

(52) U.S. Cl. 435/5; 422/38

(57) ABSTRACT

A method for the isolation or detection of a prion in a sample is disclosed. Also disclosed are methods for the disinfection and/or decontamination and/or inactivation of TSE infectivity.

This application claim priority to U.S. Application No. 60/391,895 filed Jun. 27, 2002; U.S. Application No. 60/391,897 filed Jun. 27, 2002; and U.S. Application No. 60/417,833 filed Oct. 11, 2002.

### DESCRIPTION OF THE INVENTION

The transmissible spongiform encephalopathy (TSE) diseases (scrapie in sheep, Creutzfeldt-Jakob Disease in humans, bovine spongiform encephalopathy in cattle) are lethal neurological diseases with extremely long incubation times for which there is currently no treatment. Individuals who are infected with Creutzfeldt-Jakob Disease may not have any indication of disease for decades, and might donate blood during this time. Individuals who are infected but pre-symptomatic and who die by accident or by other causes may provide organs for plantation or other medically important products. To ensure safety of the blood supply, of transplantation organs and tissues, and of other medical products obtained from human sources, a rapid, practical assay is needed which can detect the presence of TSE infectivity in blood, urine, and tissue samples. In addition, a number of domestic animals that are used for food and as a source of raw materials for biologicals, drugs, cosmetics, excipients, and other products, can also be infected with TSE diseases. These include cows, sheep, goats, farmed mink, and framed and wild deer and elk. The TSE animal diseases can infect other animals and humans. Thus, there is also a critical need for an effective method of detecting infect animals and infected animal-derived materials.

The best molecular marker for TSE disease is currently the protein called the “prion” protein, or PrP. This membrane-bound glycoprotein is found in a variety of tissues of normal host animals, in a normal conformation. In TSE-infected individuals, the conformation of some of the normal PrP protein becomes altered. This altered conformation results in a change in a number of biochemical properties of the PrP protein. For example, PrP protein becomes insoluble and forms aggregates, and it becomes more resistant to degradation by proteolytic enzymes, or proteases. The abnormal protein is called PrP^{res} because it is (partially) resistant to degradation by the enzyme Protease K, or PK, under conditions in which normal cellular PrP, PrP^{c}, is completely degraded. One current hypothesis is that the altered-conformation PrP protein is itself the infectious agent, and that it can induce conformational change in normal PrP proteins, which causes them to become infectious. In any case, the protease-resistant form of PrP, or PrP^{res}, is currently the best molecular correlate of the disease, and is generally chosen as the target for detection in biochemical assays for TSE infectivity. Any assay aimed to specifically detect PrP^{res} must be able to distinguish PrP^{res} from normal host PrP^{c}.

The most commonly used method for detection of PrP^{res} is a biochemical test based on the separation of proteins in a sample by gel electrophoresis followed by recognition of the PrP protein by a specific antibody (a method known as a “Western blot”). In this assay, PrP^{res} is distinguished from PrP^{c} by the use of PK digestion before electrophoresis to degrade PrP^{c}. Virtually every research laboratory involved in TSE research uses some version of this Western blot assay for routine detection of PrP^{res}. However, this method by itself is not sensitive enough to detect very low levels of PrP^{res}, such as the levels found in the blood of scrapie-infected hamsters, or the low levels anticipated to be found in tissues at early, pre-clinical stages of the disease, or the low concentrations that might connate human-derived or animal-derived biologicals, cosmetics, foods, and other products.

The TSE disease agent prion, or more specifically PrP^{res} is reputed to be far more resistant to disinfection by heat than viruses or bacteria. These reports have resulted in the imposition of increasingly harsh requirements for TSE disinfection in attempts to cover the liabilities associated with this resistance.

A method of inactivating or disinfecting TSE infectivity is through the use of chemical agents for relatively long exposures. These methods can destroy most biological molecules and damage equipment and delicate instruments. Further, these methods are not appropriate for use with biological materials, medical devices, surgical instruments, research instruments and equipment, raw materials, manufacturing equipment and manufactured products. They may also not be useful in various places that may need disinfection, including but not limited to manufacturing facilities, hospitals, veterinary hospitals, and necropsy and pathology labs.

The present invention is directed to a method for isolating and/or detecting a prion, in particular a prion associated with transmissible spongiform encephalopathy diseases TSEs). These proteins can be present in domestic animals used for food and used as a source for raw materials for other products. Further, to ensure the safety of the blood supply, of transplantation organs and tissues, and of other medical products obtained from human or animal sources, a rapid, sensitive, practical assay is needed which can detect the presence of TSE infectivity in blood, urine, and tissue samples and other samples.

Thus, one aspect of the present invention is a method for isolating and/or detecting a prion in a sample. This method is preferably performed utilizing an affinity resin to capture the prion protein. In one embodiment, the method utilizes a PrP-specific antibody conjugated to an affinity resin to capture the prion protein.

Thus, another aspect of the invention relates to an antibody, antigen-specific antibody fragment, or other specific binding partner, which is specific for the prion protein.

The present invention further relates to methods for the disinfection and/or decontamination and/or inactivation of TSE infectivity in a sample or material while substantially preserving the integrity of the sample or material. In one embodiment of the invention, such method makes use of brief exposures to wet heat above 100° C. In another embodiment of the invention, the sample or material to be treated is briefly exposed to a solution of an alkali metal hydroxide, e.g., sodium hydroxide (NaOH). The above-described methods provide for the decontamination or disinfection of said sample or material.

The invention also relates to a method to determine the presence of a disease condition or a susceptibility to a disease condition, wherein said condition is associated with an abnormal form of a PrP (i.e., PrP^{res}) comprising contacting a cell, tissue, cell extract or sample from a patient with an antibody which is specific for PrP and detecting the presence of the abnormal form of PrP.

The terms “PrP protein”, “PrP” and “prion protein” and like are used interchangeably herein and shall mean all forms of the PrP molecule, both the infectious particle form PrP^{res} known to cause diseases (spongiform encephalopathies) in humans and animals and the noninfectious form PrP^{c} which, under appropriate conditions is converted to the infectious PrP^{res} form.

The term “PrP^{res}” is used to refer to all infection associated forms of the PrP protein. Infectious prions infect animals and cause a prion disease “scrapie,” a transmissible, degenerative disease of the nervous system of sheep and goats, as well as “bovine spongiform encephalopathy” (BSE), or “mad cow disease”, and “feline spongiform encephalopathy” of cats. Four prion diseases are known to affect humans: (1) kuru, (2) Creutzfeldt-Jakob Disease (CJD), (3) Gerstmann-Sraussler-Scheinker Disease (GSS), and (4) fatal familial insomnia (FFI). As used herein PrP^{res} includes all forms of prion protein associated with all or any of these diseases or others in any animals and in particular in humans and domesticated farm animals.

### Detection of Prion Protein

The present invention relates to a method for concentrating and/or detecting PrP^{res} in a sample. The method utilizes a unique method of concentrating and discriminating the prion protein, PrP^{res}, using a capture technology for the prion protein, PrP^{res}. This technology allows the concentration of PrP^{res} signal from extremely dilute solutions, and the selective capture of PrP^{res} signal from complex protein mixtures in which it is otherwise not detectable due to the presence of interfering substances. Thus, this capture technology can be used to increase the sensitivity of many PrP^{res} dependent assay systems including but not limited to ELISA, Capillary Immuno-Electophoresis (CIE), and the Bayer Western Blot methods (Lee et al., J Virol Methods, 877-89 (2000); Jackman et al., Electrophoresis, 24:892-6 (2003); Schmerr et al., J Chromatogr, 802:13541 (1998)). It is especially well adapted to increasing the sensitivity of Western blot assays.

This invention uses an affinity resin column to capture and concentrate PrP from a sample before detection by Western blot. This method provides a 10,000-fold or greater improvement in sensitivity of a Western blot assay, and is capable of detecting, e.g., 0.5 to 1 fg/ml of purified recombinant protein.

As discussed above, the present invention is broadly directed to a method for isolating and/or detecting a PrP^{res} in a sample. The method of the present invention comprises i) extracting a PrP^{res} containing sample in a detergent; ii) adding a protease to digest PrP^{c} present in the sample (and optionally other proteins); iii) denaturing proteins remaining in the sample after digestion; iv) preferably diluting the denatured solution to reduce the concentration of the detergent; v) applying the diluted solution to a resin, preferably an antibody-conjugated resin; and eluting any bound protein from the resin. Detergent in the sample can also be removed from the sample by conventional methods in the art.

A PrP containing sample includes, but is not limited to, blood, plasma, a biopsy from an organ, tissue homogenate, urine or other bodily fluids, a process sample, raw materials, end products or extracts from any of the above.

In one embodiment, the detergent used to extract the PrP containing sample can be an anionic detergent. It is preferable to use sodium dodecylsulfate (SDS) or Sarkosyl. In a most preferred embodiment, the detergent is Sarkosyl.

The PrP containing sample is extracted in an amount of detergent ranging from about 0.5% to greater than 2% (w/v) preferably about 1% (w/v). In a preferred embodiment the PrP containing sample is extracted in 1% Sarkosyl.

After extracting the sample with detergent, the sample is then combined with a protease to digest proteins present in the sample. PrP^{res} proteins must be resistant to the action of the protease used.

In one embodiment, the extracted solution is digested with a protease, preferably proteinase K. In a preferred embodiment, the extracted solution is digested with proteinase K in the presence of a detergent such as 1% Sarkosyl. (The protease is added such that the concentration of protease ranges from about 0.005 to about 0.1 mg/ml. In a preferred embodiment, the protease is present in an amount of about 0.05 mg/ml.

Following digestion with a protease, the sample is denatured by any method known in the art. For example, denaturation can be carried out by chaotrophs, detergents, surfactants, alkaline pH or heating the sample or a combination thereof. Preferably, denaturation is carried out by heating the PrP containing sample in a boiling water bath for a time ranging from about 1 minute to about 10 minutes. In a preferred embodiment, the denaturation time is 5 minutes.

Before the sample can be applied to the affinity resin the amount of detergent or other denaturant in the sample is preferably reduced to allow binding between proteins and the resin. Thus, after denaturation, the concentration of detergent can be reduced by diluting the denatured solution by about 1:2 to about 1:10 fold, preferably about 2 to about 3 fold, with buffer or water.

To detect PrP^{res} in the sample, the diluted sample is applied to any resin coupled to a PrP-specific antibody such as ABX (Baker), Protein G containing resin or Protein A containing resin. In a preferred embodiment, the sample is applied to a Protein A resin conjugated to an antibody or antibody fragment that specifically binds to PrP^{res}. The volume of resin or antibody-conjugated resin is as little as 1/1000 times the volume of sample to be added. In a preferred embodiment the volume of resin is 1/100^{th} times the sample volume.

Antibodies to conjugate with the resin can be, for example, polyclonal or monoclonal antibodies. The present invention also encompasses chimeric, recombinant, single chain (e.g., U.S. Pat. No. 4,946,778), and partially or fully human antibodies, as well as Fab fragments, or the product of a Fab expression library, and fragments thereof. The antibodies can be IgM, IgG, subtypes, IgG2A, JgG1, etc. Various procedures known in the art may be used for the production of such antibodies and fragments.

For preparation of monoclonal antibodies, any technique which, provides antibodies produced by continuous cell line cultures can be used. Examples include, e.g., the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

In one embodiment, the bound PrP^{res} protein is eluted from the resin or antibody-conjugated resin by boiling the protein bound resin in a buffer, preferably containing 2% SDS. The volume of buffer can be as little as 1:1 times the resin volume. Preferably the volume of buffer is twice the resin volume.

The above-described method offers distinct advantages over existing methods for detecting low concentrations of PrP^{res}. This method can be applied to blood, highly dilute PrP^{res} solutions, and other samples of high molecular complexity. When combined with the Western blot assay, this method of concentration provides at least a 1000 fold concentration of PrP over previously used methods, allowing this method to detect a thousand-fold lower concentration of PrP^{res}. Combined with ELISA-type assays the concentration method of this invention can increase intrinsic sensitivity by 1000 fold while simultaneously increasing specificity by means of the capture step. Further, this method has been shown to be adaptable to a variety of types of biological samples, including urine.

### Wet Heat Inactivation of TSE Infectivity

This method of the invention relates to inactivating TSE infectivity using brief exposures to wet heat, preferably at temperatures above about 110° C. The present method also relates to a device for conducing experiments to measure the level of inactivation achieved by any given time and temperature combination. This invention identifies regimes of wet heat killing for TSEs that will extend the usefulness of this highly effective decontamination method to a much larger number of applications than those suitable for the much harsher conditions of the steam decontamination methods currently in use.

This invention is applicable to the disinfection of liquids and solutions including animal-derived materials such as milk and gelatin and to the decontamination of solid surfaces such as surgical instruments, or production equipment used in the manufacturing of products from animal-derived or human-derived sources.

In one embodiment, a sample or material is heated to a temperature ranging from about 110° C. to about 150° C. for a time period of less than about 5 minutes. In a preferred embodiment the time period ranges from about 4 to about 30 seconds. In another preferred embodiment, a sample or material is heated to about 140° C. for about 4 seconds.

For example, quickly heating an aqueous preparation containing a measured amount of the 263K strain of scrapie to 140° C. and holding it at that temperature for 4 seconds results in the killing of 10⁵ infectious doses (ID₅₀) of scrapie infectivity. In addition, heating an aqueous preparation of the 301V strain of BSE to 140° C. and holding it at that temperate for 4 seconds resulted in a comparable amount (10⁴ ID₅₀) of killing of BSE infectivity. This invention also recognizes the critical importance of maintaining a wet environment and preventing opportunities for the infectivity to dry, in order to achieve the best possible decontamination effect.

One possible apparatus as depicted in FIG. 1, which is a laboratory scale-down of the UHT process such as that used in the gelatin manufacturing process, is used to demonstrate the effectiveness of this invention. The apparatus can be completely filled with a liquid sample so as to eliminate all headspace and exclude all air from the stainless steel capillary. A back-pressure regulator valve prevents the hydrostatic pressure from exceeding 100 psi as the temperature is increased and the fluid expands. Thermocouples inside and immediately outside the coiled tube measure the internal temperature and external temperature respectively. The capillary coil is heated in a regulated oil bath. To minimize the ramp time of the sample, two oil baths are used, one set at about 160° C., preferably 20° C. higher than the target temperature. The sample or material is first placed in the higher temperature bath and then at an empirically determined temperature shifted to the target temperature bath. When executed properly, the high temperature exposure greatly reduces the ramp time to the target temperature without any overshoot. The internal temperature of the coil and oil baths are monitored through a digital data logger attached to a computer with various options for graphical display for timing the transition between the two oil baths and the target temperature exposure. The data logger also stores a temperature time profile for each thermocouple included in the experiment. Upon completion of the exposure, the sample is plunged into a cold water bath to bring the temperature below inactivating levels. To recover the sample, while avoiding exposure to the unions in case they entrapped any air, the capillary is cut at both ends on the capillary side of the unions and the sample expressed into a sample tube with a syringe fitted to the capillary with flexible tubing.

Infectivity before and after temperature exposure is measured by bioassay of the untreated and heat-treated material in the appropriate rodent host sensitive to the infectivity (see Example 3 below). For example, hamsters can be used to assay 263K scrapie, and mice to assay 301V BSE.

Previous work (Rohwer et al., Science 223: 600-2 (1984)), has shown that exposure to 80° C. is not inactivating for TSE infectivity, 100° C. shows some inactivation, and 121° C. is strongly inactivating. If one includes the time spent by the sample between 100° C. and 140° C. as part of the exposure and then compares the data for the scrapie and BSE inactivations (see FIG. 2), it is seen that these inactivations fall on the same line and the one log difference in effect is due to differences in total exposure during the ramp phase of the exposure. Thus, there appears to be no increased resistance to inactivation by BSE compared to scrapie as has been postulated to account for the appearance of BSE in feed supplements prepared by rendering. Moreover, the inactivation appears to be first order with an inactivation rate constant of approximately 0.75 to 0.95 log₁₀ ID₅₀/s. This is a composite value obtained from the ramp exposure as well as the 140° C. exposure. The actual inactivation rate will be different for each temperature encountered during the ramp up, with the greatest rate at the highest temperature achieved, 140° C. Thus this composite rate underestimates the inactivation rate constant 140° C. Nevertheless, recognizing that the rate at 140° C. is even greater than for k=0.75 to 0.95 log₁₀ ID₅₀/s, and that a first order rate constant of 0.75 to 0.95 log₁₀ ID₅₀/s represents a loss of approximately 1 log₁₀ID₅₀ every 2 seconds, it is readily seen that increasing the time of exposure to 140° C. steam by just a few seconds will significantly increase the killing. For example <15 seconds would be required to inactivate up to 8 logs of infectivity.

The inactivation rate is also affected by the temperature. Previous work (Science, 223: 600-2 (1984)) has shown inactivation of 7 log₁₀ID₅₀ during the 50 to 60 seconds of ramp time required to reach 121° C. in the apparatus used for those experiments. Since this was the first titration sample taken and since most of the infectivity had already been destroyed by the end of the ramp up, the actual rate of inactivation at 121° C. may be significantly greater than that indicated by the first measurement and may not be significantly slower than that obtained at 140° C. However, even if the inactivation at 121° C. is slower than that at 140° C. it still occurs in seconds rather than minutes or hours and a slightly longer exposure at 121° C. will achieve the same level of inactivation obtained at 140° C. and have the advantage of greater compatibility with a wider range of materials. For example, a plastic that melts at 140° C. but not 121° C. could be treated at the latter temperature but not the former.

This invention optimizes the time and temperature parameters for wet heat inactivation of TSE infectivity between 100° C. and 140° C., thus providing new opportunities for use with a wide range of materials and processes that have been previously considered incompatible with wet heat inactivation of TSE infectivity.

The susceptibility of TSE infectivity to inactivation by wet heat, or steam, is shown to be much greater than is generally appreciated. Effective decontamination can be achieved in a few seconds of exposure to wet heat or steam, rather than minutes to hours currently specified. However, the experiments presented here confirm and extend that result to support this application.

This invention demonstrates that parameters can be chosen that are effective in killing TSE infectivity but that will not degrade products being manufactured and that are also practical for routine steam decontamination of instruments or process equipment.

The device used to make these measurements is unique. It can be rapidly equilibrated at the target temperature to minimize the ramp time. It contains an integral thermocouple for real time measurement of the sample temperature via a computer. It contains no head space, is protected against over-pressurization, and can be assembled and loaded without contaminating the outside of the device and provides for recovery of the sample without cross-contamination.

This invention recognizes the critical importance of eliminating air and any possibility of drying, for effective wet heat inactivation.

The present method offers distinct advantages over the current specifications for wet heat inactivation of TSE infectivity (i.e. 134° C. for 18 minutes in a porous load autoclave or 132° C. for 1 or more hours in a gravity displacement autoclave) because it requires both much shorter exposures and, in some implementations, much lower temperatures for equivalent effect. This transient exposure to high temperature is short enough that many products or devices would be unharmed by the treatment. More extreme specifications for wet heat inactivation of TSEs are impractical for decontaminating many products or devices, because the products or devices would themselves be destroyed or impaired.

Any device used should be configured such that there is little or no opportunity for drying of the product prior to wet heat inactivation within the process environment. A dry-heat environment or anhydrous environment may result in the inability to inactivate the infectious agents within those environments. Furthermore, encapsulation of the infectious agent in a lipid envelope may result in the inability to inactivate the infectious agent.

A unique feature of a preferred device used to produce the data supporting this application is the lack of head space in the inactivation vessel. This design eliminates any possibility of drying the specimen before or during the inactivation.

### Alkali Inactivation of a Prion

The present invention also relates to a method for inactivating TSE infectivity by exposing a sample or material to a solution of hydroxide ions and that the hydroxide ions inactivate TSE infectivity by a process of denaturation and not by hydrolysis. This inactivation mechanism is significant because hydrolysis (i.e., breaking covalent bonds to give complete chemical degradation of a molecule) requires much harsher conditions and longer times than denaturation (i.e., changing the three-dimensional, “tertiary” structure of a molecule without breaking covalent bonds to change the “secondary” or “primary” structure of the molecule) and this mechanism can be performed using short exposure times. In one embodiment, the method is carried out at temperature ranging from about 15° C. to about 40° C. In a preferred embodiment, the method is carried out at room temperature.

In another embodiment, hydroxyl ions are added to a PrP containing sample, such as a preparation of tissue homogenate, for a period of time sufficient to allow denaturation. The denaturant can be any reagent capable of producing solutions that are 0.1N or greater in hydroxyl ions. Thus, any strong base including sodium hydroxide, lithium hydroxide and potassium hydroxide can be used. In a preferred embodiment 0.1N or greater hydroxide, e.g., sodium hydroxide, is used. The sample and denaturant are mixed for a time period ranging from about 30 seconds to about 30 minutes. Preferably, the time period ranges from about 30 seconds to about 15 minutes, more preferably from about 30 seconds to about 10 minutes, even more preferably from about 30 seconds to about 2 minutes, and most preferably less than about 2 minutes. Homogenized tissue can be prepared by any known method in the art.

After denaturation, the mixture can be neutralized by the addition of an acid. In a preferred embodiment, the acid is hydrochloric acid when the base is a met alkali base. The amount of acid added is sufficient to neutralize the base. The neutralization of the denaturant can be monitored using known methods in the art. For example, the dye phenol red, which is a pH indicator, can be used.

After neutralization, the sample can be then assayed for infectivity, by intracranial inoculation of serial dilutions into hamsters or any other appropriate host. This biological titration takes about a year for final results to be obtained. Further, the sample can be assayed for denaturation by measuring the resistance of the PrP protein to a protease such as proteinase K. If PrP protein is present and detectable by Western blot assay, but no longer resistant to proteinase K, then it is determined to have been denatured but not hydrolyzed. (The Western blot assay involves electrophoretic separation of proteins followed by detection and identification of PrP by binding to anti-PrP antibodies.). Furthermore, the sample can be assayed for hydrolysis of all prion protein, both PrP^{c} and PrP^{res}, by determining whether PrP, in any configuration, is present and detectable by the standard Western blot for PrP.

This invention will be described below by way of specific examples and appended figures, which purpose is to illustrate and not limit the scope of the invention.

### BRIEF DESCRIPTION OF THE DRAWINGS

FIG. 1 is a perspective view of apparatus assembled to collect a TSE infected sample; and

FIGS. 2-4 are views similar to FIG. 1 showing steps for handling the TSE infected sample in the apparatus after applying heat to deactivate the sample.

### DETAILED DESCRIPTION

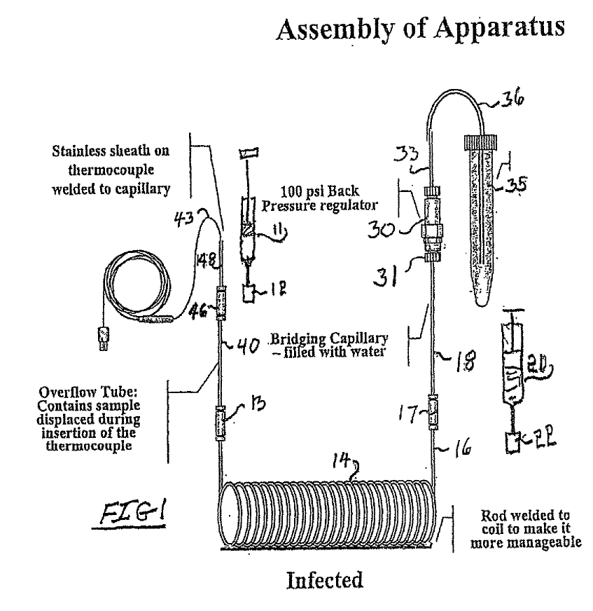

Referring now to FIG. 1, apparatus 10 for performing the method of the present invention is illustrated. To use the apparatus 10, a test sample is first prepared from a homogenate of TSE infected tissue.

The test sample is drawn into a first syringe 11 and air is cleared from the syringe. The original needle is changed to one terminating in a union 12 and the union is attached to a first coupling 13 at a first end of a capillary storage tube configured as a coil 14. The test TSE sample is injected into the coil 14 and exits through a flexible tube 16 attached to the coil by a second coupling 17 and flows to a capillary receiving tube 18 for collecting excess TSE sample.

The capillary receiving tube 18 is fitted with a union at one end which is initially attached to a second syringe 21 containing water and having a needle with a union 22 attached to the needle. The tube 18 is filled with water from the second syringe 21 through the union 22 and the capillary 18 fills the coil 14 through its union 17. A 100 psi back pressure regulator 30 is attached to the free end of capillary tube 18 by a connector 31 and a short length of capillary 33 is attached to the low pressure side of the back pressure regulator 30. A trap 35 is connected to the capillary 33 through a short length of flexible tubing 36 and be is used to secure the trap 35 to the pressure regulator 30.

The first syringe 11 used to fill the coil 14 with the infected sample from the first coupling 13 is removed, and in its place is attached to the first coupling 13, an unfilled length of capillary 40. The capillary 40 has the exact length to contain all of the TSE sample that will be displaced from the coil 14 during insertion of a thermocouple sensor lead 43.

A union ferrule 46, backed by a nut, is swaged onto a short length of capillary 48 and the sinless steel-sheathed thermocouple wire 43 is slid through the capillary 48 so that a predetermined length protrudes sufficient to place the sensor at the end of the thermocouple lead in the first loop of the capillary coil 14. The joint between the capillary 48 and the sheath of the thermocouple lead 43 is sealed with a silver solder weld 51.

The sensor end of the stainless steel sheathed thermocouple lead 43 is slid into the still empty capillary 14, through the first coupling 13 and into the capillary coil 14. Since the coil 14 is filled with infected TSE sample, the infected TSE sample is displaced into capillary 40 as the thermocouple lead 43 is inserted. By watching the orifice at the insertion point 51, one can see the gelatin rise to within a millimeter or less of the end of the capillary 48 just before completing the insertion. The assembly of the apparatus 10 is now complete.

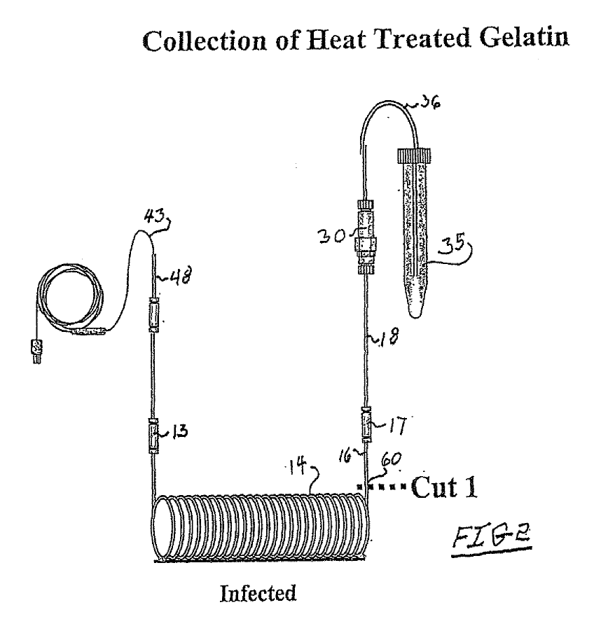

Referring now to FIG. 2, after the thermal pulse is applied to the capillary tube coil 14 to sterilize the infected TSE sample, the capillary coil tubing at the end thereof connected to the back pressure regulator 30 is straightened by bending adjacent the second coupling 17 and clamped in a small vice. The capillary coil tubing is then carefully cut in front of the second coupling 17 with a triangular file, working the file slowly around the tubing to keep the new cut flat and straight. The tubing is then cut at location 60 below the second coupling 17 to minimize any dilution of the collected sample with the water in the transition tube 18 between the coil 14 and the back pressure regulator 30. The file is discarded after use.

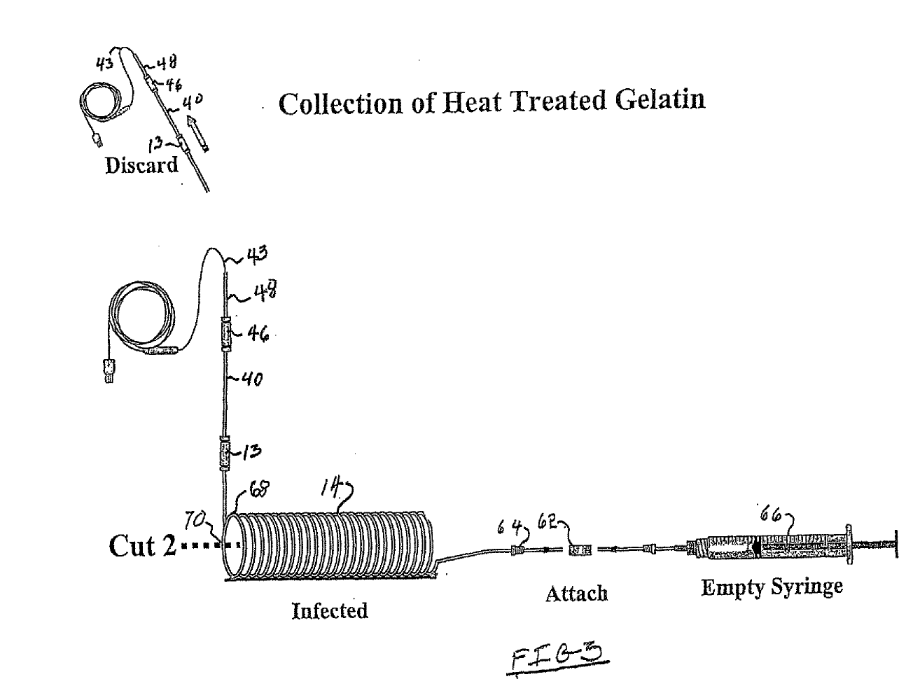

Referring now to FIG. 3, the cut end of the tubing 16 at 60 is fitted with a new nut 62 and ferrule 64 and attached to an empty syringe 66 that will be used to push the infected TSE sample from the coil 14 with air. The thermocouple end 68 of the coil 14 is then straightened, is clamped in a vice and cut with a file at location 70. The thermocouple wires 43 are then pulled from the capillary tube and discarded.

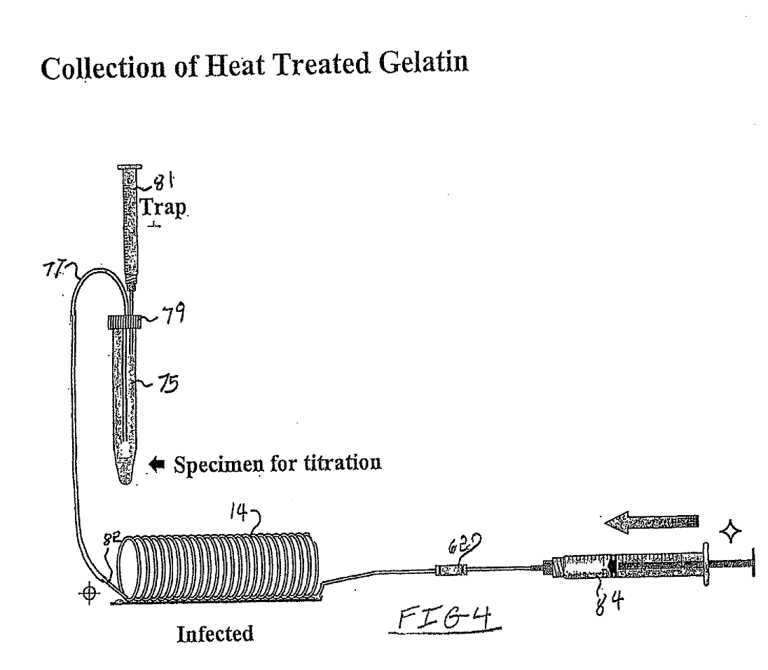

As is seen in FIG. 4, a sample of the TSE material is then collected in a 15 ml conical centrifuge tube 75 which fitted with a length of flexible tubing 77 through a hole in its cap 79 that is bored and slightly smaller than the OD of the flexible tubing. An aerosol trap consisting of a 1 ml syringe 81 packed with Kimwipes is also fitted to the cap 79. The flexible tubing 77 is then attached to the cut end of the capillary coil 14 by a friction fit. The TSE sample is then displaced from the coil 14 into the centrifuge tube 75 by air injected from a third syringe 84 into the opposite end of the coil 14 through ferrule 64.

The infectivity in the expressed TSE sample in the centrifuge tube 75 is then assayed by end point dilution titration in hamsters or mice depending upon the TSE strain being inactivated an the resulting titer compared to the pretreatment value to obtain the level of inactivation.

In the foregoing and in the following examples, all temperatures are set forth uncorrected in degrees Celsius; and, unless otherwise indicated, all parts and percentages are by weight.

### EXAMPLES

### Example 1

### Capture of PrP^{Res} from a Sample

Resin Preparation

The affinity resin is prepared according to the instructions of the manufacturer (Pierce). 3F4 antibody is coupled to a Protein A gel matrix and covalently bound to the resin by cross-linking. Extensive washing with increasingly stringent conditions, from PBS to glycine buffer at pH 2.5 to 0.1% SDS, removes non-covalently bound 3F4 antibody molecules and thus reduces the background in the Western blot.

Resin Application to Capture PrP^{Res}

PrP^{res} extraction—PrP^{res} forms highly aggregated and insoluble fibrillary structures. PrP^{res} is extracted into solution for efficient protein presentation to the affinity resin. Extraction is accomplished by incubating the PrP^{res}-containing material with a strong ionic detergent, such as 1% Sarkosyl.

PK digestion—Since 3F4 antibody does not distinguish between normal PrP and disease-associated PrP^{res}, selection for PrP^{res} occurs before the capture step. Selection is achieved by incubating the sample with proteinase K (PK) or other proteases, under conditions that destroy PrP^{c} but not PrP^{res}. The presence of Sarkosyl did not affect PK digestion. PK degrades normal PrP and partially cleaved PrP^{res}. PrP^{res} is detected and captured by the 3F4 antibody-resin.

PrP^{res} denaturation —The 3F4 antibody has a higher affinity for denatured PrP^{res} than for native PrP^{res}. In order to obtain efficient denaturation, the solution containing PrP^{res} is heated in a boiling water bath for 5 minutes. The presence of Sarkosyl improves solubilization of boiled PrP^{res} and keeps the protein in solution. However, after PrP^{res} denaturation, the detergent is diluted about 2-3 fold to allow efficient binding of 3F4 antibody affinity resin to PrP^{res}.

PrP^{res} capture and elution—About 1 to 10 ml of PrP^{res}-containing solution is incubated overnight with 10 μl of resin. Larger volumes can be scaled proportionately. As much as 100 μg of brain-derived PrP^{res} can be bound to 10 μl of resin. However, this method can also be applied to very dilute solutions containing less than 10 ng of PrP^{res} in 10 mls. After incubation, the solution is centrifuged and the unbound material is removed by pipetting off the supernatant. The resin is washed extensively and eluted by boiling with 20 μl of SDS-PAGE loading buffer containing 2% SDS.

Maximum sensitivity is obtained by loading the eluate in one single lane of an SDS-PAGE gel, followed by electrophoresis and detection by Western blot using 3F4 antibody. Detection can be by any primary or secondary antibody detection system.

### Example 2

### NaOH Incubation and Neutralization

### Materials:

### Preparation of Brain Homogenates:

Brain is homogenized by sonication to a final concentration of 10% in buffer.

Temperature: Brain homogenate from scrapie-infected hamsters and NaOH is pre-equilibrated to 20° C. 10× phosphate and the acid are pre-equilibrated to 0° C. in an ice bath, to counteract the heating that occurs when acid is added to a base such as NaOH. Pilot experiments have shown that adding the phosphate and acid at room temperature produces only a slight warming of the mixture.
Transfer: There is concern that minute amounts of brain homogenate (which would still contain significant infectivity) might deposit on the upper walls of the vessel where they would escape contact with NaOH and thereby give a false indication of survival. To avoid this, the entire sample is transferred by pipet to a fresh vessel immediately after the NaOH is added. Great care is taken not to contact the walls of the new vessel with the pipet. However, even if there is undetected splashing onto the walls of the now vessel the splashed droplets contain NaOH.
Addition of reagents: Each addition of reagent is measured into a polypropylene tube, and the contents are poured into the reaction vial at the appropriate times. Pouring required 2 to 3 seconds to complete without splashing.
Phenol red: Control experiments are conducted with all reagents except infected brain homogenate, and using a pH meter as a second monitor of pH, to establish how the phenol dye behaves in this reaction mixture. In these control experiments, the deep pinkish purple of the phenol red in the NaOH mixture remains unchanged with the addition of acid, until the pH is 7.0±0.5, at which point the dye suddenly lightens to a less intense, lighter pink. One or two more drops of acid turns the dye yellow, but the pH still remains within 0.5 units of 7.0. The dye becomes distinctly yellow as the pH drops to 6.0 with further addition of acid, and is lemon yellow at pH 5.0. Below pH 5.0, the dye turns a peachy orange. In conclusion, these control experiments, as well as the reproducibility of the results, indicate that phenol red gives a reliable and accurate measure of the pH.

In three determinations on all reagents except infected brain homogenate, the reaction requires 16.1 ml of 1.25 M hydrochloric acid (HCI) to reach neutrality (pH 7.6) as determined by the color of the phenol red.

### Detailed Procedure:

1. Ten (10) ml of 2N NaOH are measured by pipet and transferred into a 15 ml polypropylene centrifuge tube. The tube and contents are allowed to equilibrate in a water bath to 20° C.

2. Four (4) ml of 10× phosphate buffer (1M sodium phosphate buffer, pH 7.0)+10× phenol red are measured by pipet and transferred into a second 15 ml polypropylene centrifuge tube. The tube and contents are allowed to equilibrate in a water-ice bath to 0° C.

3. Sixteen (16) ml of 1.25 M HCI are measured by pipet and transferred to a 50 ml polypropylene centrifuge tube. The tube and contents are allowed to equilibrate in a water-ice bath to 0° C.

4. Ten (10) ml of 10% scrapie-infected brain, homogenized in phosphate buffered saline (PBS), pH 7.2, are added to a 120 ml flat-bottom polypropylene snap-cap vial, containing a star-shaped magnetic stirbar. The tube and contents are allowed to equilibrate to 20° C. The stirring motor is turned on slowly to minimize splashing.

5. At t=0, the pre-measured 10 ml of 2N NaOH is poured rapidly into the 120 ml vial containing scrapie brain homogenate. This procedure takes less than 3 seconds.

6. At t=15 seconds, the contents of the vial are transferred to an identical 120 ml flat bottom vial+stirbar, using a 25 ml pipet. Stirring is continued.

7. At t=110 seconds, the pre-measured 4 ml of phosphate buffer-phenol red are poured into the vial.

8. At t=120 seconds, the pre-measured 16.0 ml of 1.25 N HCI is poured into the vial.

9. At t=123 seconds, 1.25 N HCI is added drop-wise until the phenol red just turns yellow.

10. Steps 1-7 are repeated three times with a new sample each time. The NaOH exposure is stopped by HCI addition (Steps 8+9) at 2 minutes, 5 minutes, 15 minutes, and 30 minutes, respectively.

### Bioassay by Titration in Hamsters

Samples of brain homogenate incubated with 1N NaOH for varying times and then neutralized, as described above, are titrated for infectivity by intra-cerebral inoculation into hamsters, following standard procedures as described in the Manual of Standard Operating Procedures for the Laboratory of Molecular Neurovirology. Samples of the same preparation of brain homogenate that are not treated with NaOH are also titrated in hamsters at the same time. Results for the NaOH-treated and untreated samples indicate that exposure to NaOH for only 2 minutes inactivated 99.999% of the infectivity.

Determination of Denaturation Vs. Hydrolysis

Samples of brain homogenate incubated with 1N NaOH as above are divided into two aliquots. One aliquot is digested with the protease Proteinase K, and the second aliquot is untreated. These aliquots are then assayed for the presence of the PrP protein by the PrP Western blot, following standard procedures. In this assay, detection of PrP in the aliquot, which is not treated with Proteinase K, indicates the total amount of PrP present (both “normal” host PrP^{c} and infection-specific PrP^{res}). A decrease in this amount of PrP in the treated aliquot indicates that the PrP protein has been chemically degraded, or hydrolyzed. Detection of PrP in the aliquot, which is digested with Proteinase K, indicates the amount of protease-resistant, infection-specific PrP. A decrease in this amount indicates that the amount of PrP^{res} has been reduced, which could be accomplished by simple denaturation of the protein.

After only 2 minutes of exposure to NaOH the amount of PrP^{res} is significantly reduced, while the amount of total PrP is not detectably reduced. This result indicates that the brief exposure to NaOH denatures the PrP^{res} but does not hydrolyze it. With increasing time of exposure to NaOH, the total amount of PrP^{res} detected in the Western blot assay declines, to about a hundred fold reduction after 15 minutes. This indicates that the PrP^{res} is being hydrolyzed by the NaOH, but the time course for hydrolysis is slower than that for denaturation.

The results of the titration and denaturation vs. hydrolysis experiments are summarized in FIG. 3. The graph shows that the time course of inactivation of infectivity follows the same time course as denaturation, and that hydrolysis is much slower and less complete.

The short exposure times involved in this invention would allow NaOH to be used in a variety of applications where longer exposures would not be feasible, because the product, material, or equipment being decontaminated could not withstand long exposure to the caustic action of concentrated NaOH, or because the opportunity for disinfection would not allow long contact with NaOH.

Concentrations of NaOH as low as 0.1N are also effective, again allowing its use in applications where longer exposures would destroy the product, material, or equipment being decontaminated.

The inactivation by NaOH can be achieved even faster by increasing the temperature of exposure to 60° C. or 80° C. or higher.

This invention is not specific to NaOH but will work equally well with any reagent capable of producing solutions that are 0.1N or greater in hydroxyl ions. As an example, potassium hydroxide (KOH) would be equally effective. However, calcium hydroxide (CaOH₂) would not be as useful.

It is useful to point out that the end product of the treatment, NaCl (sodium chloride), is not a hazardous material. Most salts that would be formed upon the neutralization of the base with the acid would not be a hazardous material.

### Example 3

### Bioassay for Infectivity of a Wet Heat Treated Sample

The bioassay consists of a standard end-point dilution titration cared out in rodents. Briefly, a series of 10 fold dilutions of the treated material are prepared. In the case of scrapie, 50 μl of each dilution is inoculated intra-cerebrally (IC) into each of four hamsters which are maintained for 18 months (540 days). In the case of BSE, five mice are each inoculated with 30 μl of each dilution and held for 20 months (600 days). Animals are checked daily and scored twice weekly for symptoms of disease. Once symptoms are noted, animals are scored daily. Disease is diagnosed clinically by the characteristic progression of symptoms for scrapie in hamsters or BSE in mice. Disease state is confirmed biochemically by Western blot assay for PrP^{res} in the brain tissue from hamsters or mice, after death.

The topic headings set forth above are meant as guidance as to where certain information can be found in the application. They are not intended to be the only source in the application where information on such a topic can be found.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated in their entirety by reference.

What is claimed is:

1. A method for decontaminating or disinfecting a TSE infected sample or material comprising heating the sample or material in a wet environment to a temperature above about 100° C. for a time period of less than about 1 minute, wherein the integrity of said sample or material is substantially preserved.

2. The method of claim 1, wherein the sample or material is heated to a temperature above about 121° C.

3. The method of claim 1, wherein the sample or material is heated to a temperature of about 100° C. to about 150° C.

4. The method of claim 1, wherein the sample or material is heated to a temperature of about 100° C. to about 140° C.

5. The method of claim 1, wherein the time period is less than about 30 seconds.

6. The method of claim 1, wherein the time period is about 3 to about 15 seconds.

7. The method of claim 1, wherein the time period is about 4 seconds.

8. The method of claim 1, wherein the decontaminating or disinfecting is performed substantially in the absence of air.

9. The method of claim 1, wherein the sample is heated to about 140° C. for about 4 seconds.

10. A method for decontaminating or disinfecting or inactivating a TSE infectivity in a sample or material comprising contacting the sample or material with a solution of about 0.1N or greater alkali hydroxide ions for a time period ranging from about 30 seconds to about 10 minutes to decontaminate or disinfect or inactivate the infectivity in said sample or material.

11. The method of claim 10, wherein said alkali hydroxide is sodium hydroxide.

12. The method of claim 10, wherein said sodium hydroxide is from about 0.1N to about 2N.

13. The method of claim 10, wherein the time period is less than about 2 minutes.

14. The method of claim 10, wherein the time period is about 30 seconds to about 2 minutes.

15. The method of claim 10, wherein the time period is about 30 seconds.

16. The method of claim 15, wherein said solution is about 1N sodium hydroxide.

17. The method of claim 10, wherein the method is carried out at about room temperature.

18. The method of claim 10, wherein said solution is about 0.1N or higher sodium hydroxide and said time period is less than about 2 minutes.

19. A method for decontaminating, inactivating or disinfecting a TSE infectivity in a sample or material comprising:
i) contacting the sample or material with a solution of about 0.1 N or greater alkali hydroxide ions for a time period ranging from about 30 seconds to about 10 minutes; and
heating the sample or material in a wet environment to a temperature above about 100° C. for a time period sufficient to decontaminate, inactivate or disinfect said sample or material.

20. A method for decontaminating, inactivating or disinfecting a TSE infectivity in a sample or material comprising
i) contacting the sample or material with a solution of alkali hydroxide ions; and
heating the sample or material in a wet environment to a temperature above about 100° C. for a time period of less than about 1 minute to decontaminate, inactivate or disinfect said sample or material.

21. A method for detecting or isolating a PrP^{res} signal from a sample comprising:
i) adding a protease in the presence of detergent to a PrP^{res} containing sample to digest PrP^{c} present in the sample but not PrP^{res};
ii) denaturing PrP^{res} remaining in the sample after digestion;
iii) applying the resultant solution to a resin; and
iv) eluting the PrP^{res} signal from the resin.

22. The method of claim 21, wherein prior to adding a protease, the sample is extracted with an ionic detergent.

23. The method of claim 22, wherein the ionic detergent is SDS or Sarkosyl.

24. The method of claim 22, wherein the amount of detergent is as little as 0.1%.

25. The method of claim 21, wherein the protease is ______, ______, ______ or proteinase K.

26. The method of claim 21, wherein denaturing is performed by boiling.

27. The method of claim 21, wherein prior to applying the solution to a resin, the solution is diluted.

28. The method of claim 21, wherein the resin is Protein G or Protein A.

29. The method of claim 21, wherein the resin is Protein A conjugated to an anti-PrP antibody.

30. The method of claim 21, wherein a Western blot is performed after the eluting step.

31. The method of claim 10 wherein the integrity of said sample or material is substantially preserved.

32. The method of claim 19 wherein the integrity of said sample or material is substantially preserved.

33. The method of claim 20 wherein the integrity of said sample or material is substantially preserved.

34. Apparatus for decontaminating or disinfecting a TSE infected sample material, comprising
a capillary storage tube having a first end with a it coupling and a second end with a second coupling;
a first syringe for collecting a sample of TSE infected material through a first needle, the first needle being removable and replaceable by a tube terminating in a needle coupling connectable to the first coupling of the capillary storage tube for injecting the TSE sample into the capillary storage tube;
a bridging capillary connected to the second coupling for receiving excess sample;
a second syringe for filling with water and for attachment to the bridging regulator capillary;
a back pressure regulator for attachment to the bridging regulator capillary after the storage capillary has been charged with water from the second syringe;
a trap attachable to the low pressure side of the back pressure regulator;
an overflow capillary tube of a length to contain a displaced portion of the TSE sample, the overflow capillary tube being coupled to the first coupling upon disconnecting the first syringe therefrom, the overflow capillary tube having a third coupling, and
a thermocouple for attachment to the third coupling of the overflow capillary tube; the thermocouple having a sensor lead extending through the overflow capillary tube and into a portion of the capillary storage tube until the TSE sample rises to a preselected level in the overflow capillary tube, wherein the thermocouple senses the temperature of the TSE sample in the capillary storage tube upon applying a heat pulse to the capillary storage tube to disinfect the TSE infected sample materials in the capillary storage tube.

35. The apparatus of claim 34 wherein the capillary storage tube is configured as a coil.

36. The apparatus of claim 35 wherein the capillary storage tube configured as a coil is stabilized by an axially extending rigid rod welded thereto.
